(19) United States
(12) Patent Application Publication
(10) Pub. No.: US 2014/0073062 A1
(43) Pub. Date: Mar. 13, 2014

(54) SPECIMEN SOLUTION ASSAY DEVICE, SPECIMEN SOLUTION ASSAY METHOD, AND IMMUNOCHROMATOGRAPHIC SENSOR DEVICE

(71) Applicant: TOKYO ELECTRON LIMITED, Minato-ku (JP)

(72) Inventor: Akitake TAMURA, Tsukuba-city (JP)

(73) Assignee: TOKYO ELECTRON LIMITED, Minato-ku (JP)

(21) Appl. No.: 14/023,999

(22) Filed: Sep. 11, 2013

(30) Foreign Application Priority Data
Sep. 11, 2012 (JP) 2012-199475

(51) Int. Cl. G01N 33/543

(52) U.S. Cl. CPC G01N 33/54366
USPC 436/501; 422/70

(57) ABSTRACT

A specimen solution assay device includes a specimen solution dropping device which drops a specimen solution sequentially onto each of sample pads of immunochromatographic sensors positioned adjacent to each other in a transverse direction of each of the immunochromatographic sensors, and an image information acquisition device which acquires image information of a test area of each of the immunochromatographic sensors onto which the specimen solution is dropped by the specimen solution dropping device.

### CROSS-REFERENCE TO RELATED APPLICATIONS

The present application is based upon and claims the benefit of priority to Japanese Patent Application No. 2012-199475, filed Sep. 11, 2012, the entire contents of which are incorporated herein by reference.

### BACKGROUND OF THE INVENTION

1. Field of the Invention

Various aspects and embodiments of the present invention relate to an assay device for a specimen solution and an assay method for a specimen solution.

2. Description of Background Art

Detection techniques exist which detect the presence of a target substance serving as a detection target. For example, there is a technique that uses an immunochromatographic sensor as the detection technique. In the immunochromatographic sensor, a sample pad, a conjugate pad, a test line, a control line, and an absorption area are arranged in that order in a longitudinal direction. Referring to Japanese Unexamined Patent Publication No. 2012-73125, in a detection process using the immunochromatographic sensor, a user drops a specimen solution onto a sample pad and detects the presence of a target substance based on whether a test line is colored after that. The entire contents of this publication are incorporated herein by reference.

### SUMMARY OF THE INVENTION

According to one aspect of the present invention, a specimen solution assay device includes a specimen solution dropping device which drops a specimen solution sequentially onto each of sample pads of immunochromatographic sensors positioned adjacent to each other in a transverse direction of each of the immunochromatographic sensors, and an image information acquisition device which acquires image information of a test area of each of the immunochromatographic sensors onto which the specimen solution is dropped by the specimen solution dropping device.

According to another aspect of the present invention, an immunochromatographic sensor device includes a sheet, and immunochromatographic sensors positioned adjacent to each other in a transverse direction of the sheet. Each of the immunochromatographic sensors includes a sample pad onto which a specimen solution is dropped and a test area which tests the presence of a target substance in the specimen solution dropped onto the sample pad.

According to yet another aspect of the present invention, an assay method includes dropping a specimen solution sequentially onto each of sample pads of immunochromatographic sensors which are positioned adjacent to each other in a transverse direction of each of the immunochromatographic sensors, and acquiring image information of a test area of each of the immunochromatographic sensors onto which the specimen solution is dropped.

### BRIEF DESCRIPTION OF THE DRAWING

A more complete appreciation of the invention and many of the attendant advantages thereof will be readily obtained as the same becomes better understood by reference to the following detailed description when considered in connection with the accompanying drawings, wherein:

FIG. 1 is a block diagram illustrating an example of the structure of an assay device according to one embodiment;

FIG. 2 is a diagram illustrating an example of an immunochromatographic sensor according to a first embodiment;

FIG. 3 is a diagram illustrating an example of the immunochromatographic sensor according to the first embodiment;

FIG. 4 is a flowchart illustrating an example of a flow of processing performed by the assay device according to the first embodiment;

FIG. 5 is a block diagram illustrating an example of the structure of an assay device according to a second embodiment; and

FIG. 6 is a diagram illustrating a process of forming an immunochromatographic sensor using a formation control unit according to the second embodiment.

### DETAILED DESCRIPTION OF THE EMBODIMENTS

The embodiments will now be described with reference to the accompanying drawings, wherein like reference numerals designate corresponding or identical elements throughout the various drawings.

### First Embodiment

In an embodiment of the present invention, an assay device includes a dropping unit which drops a specimen solution sequentially onto each of the sample pads of multiple immunochromatographic sensors arranged adjacent to each other in a transverse direction, and an acquisition unit which acquires image information of a test area of each immunochromatographic sensor onto which the specimen solution is dropped by the dropping unit.

In an example of the embodiment, the assay device further includes a detection unit which detects the presence of a target substance based on the image information acquired by the acquisition unit.

In another example of the embodiment, the immunochromatographic sensor includes the sample pad, a conjugate pad, the test area, a control line and an absorption area which are arranged in this order in a longitudinal direction, and multiple immunochromatographic sensors are arranged on a sheet in a transverse direction of the immunochromatographic sensors.

In yet another example of the embodiment, the sheet has an intersensor area between the immunochromatographic sensors adjacent to each other in a transverse direction, the intersensor area being an area where the movement speed of the specimen solution in a transverse direction is relatively slow compared with the movement speed in a longitudinal direction.

In yet another example of the embodiment, the sheet has a hydrophobic portion or a break as the intersensor area.

In yet another example of one embodiment, an assay method includes dropping a specimen solution sequentially onto each of the sample pads of multiple immunochromatographic sensors arranged adjacent to each other in a transverse direction, and acquiring image information of a test area of each immunochromatographic sensor onto which the specimen solution is dropped.

### Structure of Assay Device According to First Embodiment

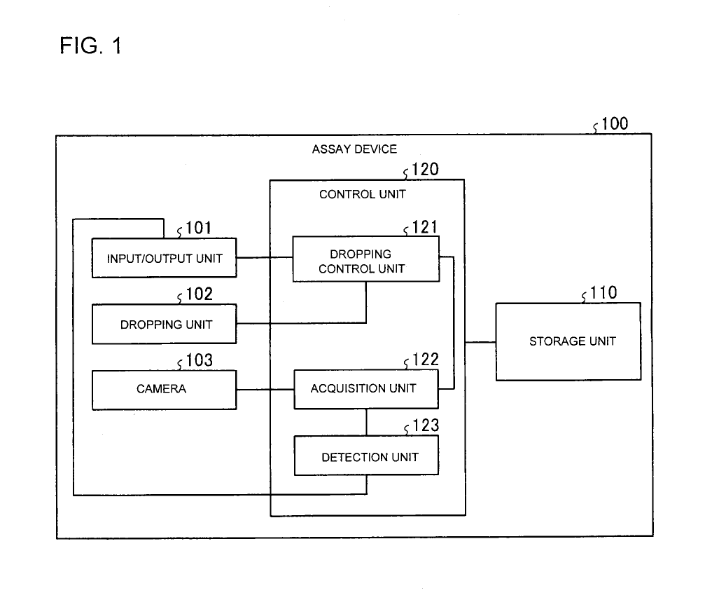

FIG. 1 is a block diagram illustrating an example of the structure of an assay device according to the first embodiment. In the example illustrated in FIG. 1, an assay device 100 includes an input/output unit 101, a storage unit 110, and a control unit 120.

Input/output unit 101 is connected to control unit 120. Input/output unit 101 receives information and directions from a user, and inputs the received information and directions into control unit 120. Input/output unit 101 receives information from control unit 120 and outputs the received information. Input/output unit 101 is a keyboard and mouse, a microphone, a display, a speaker, etc. The information and directions received by input/output unit 101, and the information output from input/output unit 101, are described in detail below.

A dropping unit 102 drops a specimen solution onto an immunochromatographic sensor. Specifically, dropping unit 102 drops the specimen solution sequentially onto each of the sample pads of multiple immunochromatographic sensors by being controlled by control unit 120.

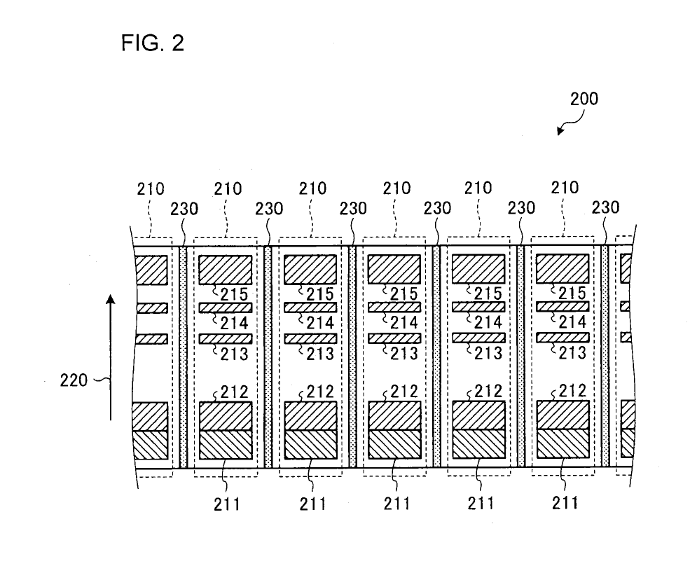

FIG. 2 is a diagram illustrating an example of the immunochromatographic sensor according to the first embodiment. In the first embodiment, multiple immunochromatographic sensors 210 arranged adjacent to each other in a transverse direction are formed on a sheet 200. In the example illustrated in FIG. 2, each of multiple immunochromatographic sensors 210 arranged adjacent to each other in a transverse direction includes a sample pad 211, a conjugate pad 212, a test area 213, a control line 214, and an absorption area 215 which are arranged in that order in a longitudinal direction. In the example illustrated in FIG. 2, multiple immunochromatographic sensors 210 are arranged in a transverse direction of the immunochromatographic sensor 210 on sheet 200.

In the immunochromatographic sensor 210, the specimen solution is dropped onto sample pad 211. Conjugate pad 212 contains a substance which specifically binds to a target substance serving as a detection target, and the substance is a labeled substance. Conjugate pad 212 contains, for example, a gold colloid labeled antibody. An antibody is immobilized on test area 213 in advance. The antibody immobilized on test area 213 specifically binds to a complex of the target substance and the labeled substance contained in conjugate pad 212. The antibody is immobilized on control line 214 in advance. The antibody immobilized on control line 214 specifically binds to the labeled substance contained in conjugate pad 212. Absorption area 215 absorbs the specimen solution.

As indicated by arrow 220 of FIG. 2, when the specimen solution is dropped onto sample pad 211 in immunochromatographic sensor 210, the specimen solution spreads in a longitudinal direction of immunochromatographic sensor 210. The specimen solution spreads on sheet 200 in a longitudinal direction of immunochromatographic sensor 210 by capillarity action, for example. The specimen solution dropped onto sample pad 211 subsequently comes into contact with the labeled substance by way of conjugate pad 212, and the target substance and the labeled substance specifically bind to each other when the target substance is contained in the specimen solution. When the specimen solution subsequently arrives at test area 213, when the target substance is contained in the specimen solution, and when the target substance and the labeled substance have specifically bound to each other, the complex of the labeled substance and the target substance specifically binds to the antibody in test area 213, and test area 213 is colored. Then, if the specimen solution subsequently arrives at control line 214, the labeled substance and the antibody immobilized on control line 214 specifically bind to each other, and control line 214 is colored. Subsequently, the test solution is absorbed by absorption area 215 when the specimen solution spreads by the capillarity action.

Because of this arrangement in which multiple immunochromatographic sensors 210 are formed adjacent to each other on the sheet 200, immunochromatographic sensors 210 are used consecutively.

As illustrated in FIG. 2, sheet 200 has an intersensor area 230 between immunochromatographic sensors 210 adjacent to each other in a transverse direction. The intersensor area 230 is an area where the movement speed of the specimen solution in a transverse direction is relatively slow as compared with the movement speed in a longitudinal direction. For example, sheet 200 has a hydrophobic portion or a break as intersensor area 230. As a result, in sheet 200, the specimen solution dropped onto sample pad 211 is prevented from affecting other immunochromatographic sensor 210.

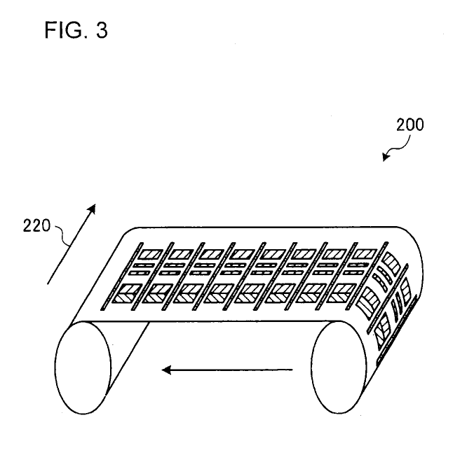

FIG. 3 is a diagram illustrating an example of the immunochromatographic sensor according to the first embodiment. As illustrated in FIG. 3, sheet 200 may be wound in the form of a roll. For example, immunochromatographic sensor 210 may be wound in the form of a roll before the specimen solution is dropped by dropping unit 102, or the immunochromatographic sensor 210 may be wound in the form of a roll after a camera 103 has completely photographed the image information of immunochromatographic sensor 210.

A description about FIG. 1 will be made again. Camera 103 photographs the image information of test area 213 of immunochromatographic sensor 210 onto which the specimen solution is dropped by dropping unit 102. The image information photographed by camera 103 is acquired by control unit 120 and is used accordingly. The image information photographed by camera 103 may be still image information or moving image information.

Input/output unit 110 is connected to control unit 120. Storage unit 110 stores data used for various kinds of processing performed by control unit 120. Storage unit 110 is a semiconductor memory device, such as RAM (Random Access Memory), ROM (Read Only Memory), or flash memory, for example. Alternatively, storage unit 10 may be a hard disk, an optical disc, or the like.

Control unit 120 is connected to input/output unit 101, dropping unit 102 and camera 103. Control unit 120 includes an internal memory which stores a program that specifies the procedure of various kinds of processing, and controls the various kinds of processing. Control unit 120 is an ASIC (Application Specific Integrated Circuit), an FPGA (Field Programmable Gate Array), a CPU (Central Processing Unit), an MPU (Micro Processing Unit), or the like, for example. In the example illustrated in FIG. 1, control unit 120 includes a dropping control unit 121, acquisition unit 122, and detection unit 123.

Specifically, dropping control unit 121 drops a specimen solution sequentially onto each of the sample pads 211 of multiple immunochromatographic sensors 210 by being controlled by control unit 121. Specifically, dropping control unit 121 causes the specimen solution to be dropped sequentially onto each of sample pads 211 by controlling dropping unit 102.

For example, dropping control unit 121 drops the specimen solution sequentially onto each of sample pads 211 upon receipt of instructions from an operator.

For example, when a position onto which the specimen solution is to be dropped by dropping unit 102 is fixed, dropping control unit 121 moves sheet 200 each time the specimen solution has been dropped onto sample pad 211 of immunochromatographic sensor 210 so that sample pad 211 of the subsequent immunochromatographic sensor 210 will be in a position where the specimen solution is to be dropped by dropping unit 102. Then, the specimen solution is dropped from dropping unit 102. Dropping control unit 121 repeats the same procedure so that the specimen solution is dropped sequentially onto each of multiple immunochromatographic sensors 210 adjacent to each other in a transverse direction.

In addition, for example, when the position of sheet 200 is fixed beforehand, dropping control unit 121 moves dropping unit 102 each time the specimen solution has been dropped onto sample pad 211 of immunochromatographic sensor 210 so that sample pad 211 of the subsequent immunochromatographic sensor 210 will be in a position where the specimen solution is to be dropped by dropping unit 102. Then, the specimen solution is dropped from dropping unit 102. Dropping control unit 121 repeats the same procedure so that the specimen solution is dropped sequentially onto multiple immunochromatographic sensors 210.

Acquisition unit 122 acquires image information of test area 213 of the immunochromatographic sensor 210 onto which the specimen solution is dropped by dropping unit 102. Specifically, acquisition unit 122 acquires image information of each immunochromatographic sensor 210 onto which the specimen solution is dropped sequentially by dropping control unit 121.

For example, acquisition unit 122 acquires the image information of test area 213 photographed by camera 103 by controlling camera 103. Here, in immunochromatographic sensor 210, it takes time for the specimen solution to arrive at test area 213 or at control line 214 after the specimen solution is dropped onto sample pad 211. Based on this, a period of time until the specimen solution arrives at test area 213 or control line 214 is preset, and acquisition unit 122 acquires the image information of test area 213 of immunochromatographic sensors 210 for which the period of time, which is preset, has passed since the specimen solution is dropped.

When acquisition unit 122 acquires the image information of test area 213, acquisition unit 122 may also acquire the image information of control line 214. For example, acquisition unit 122 may acquire both the image information of test area 213 and the image information of control line 214 which may be included in the image information photographed by camera 103. This facilitates detection processing by detection unit 123.

Detection unit 123 detects the presence of a target substance based on the image information acquired by acquisition unit 122. Specifically, detection unit 123 determines whether test area 213 is colored, based on the image information. For example, a threshold of a pixel value is preset and detection unit 123 determines whether the pixel value of test area 213 is equal to or greater than the threshold. When the pixel value is determined to be equal to or larger than the threshold, it means there is target substance. Conversely, when the pixel value is determined to be smaller than the threshold, it means there is no target substance.

When control line 214 is included in the image information, detection unit 123 may detect the target substance using a pixel value of control line 214 as well as the pixel value of test area 213. For example, detection unit 123 may acquire the pixel value of control line 214 and determine whether test area 213 is colored based on the acquired pixel value.

Detection unit 123 outputs a detection result to the user via input/output unit 101. For example, detection unit 123 outputs the result on which immunochromatographic sensor 210 has detected the target substance. As a result, the user can easily and simply grasp at which time point the target substance is detected in the detection process continuously performed on multiple immunochromatographic sensors 210 adjacent to each other in a transverse direction. In this case, detection unit 123 may chronologically output the image information acquired by acquisition unit 122 along with the time points at which the image information is acquired, and may output the image information about the immunochromatographic sensor 210 by which the target substance is detected.

### Flow of Processing by Assay Device According to First Embodiment

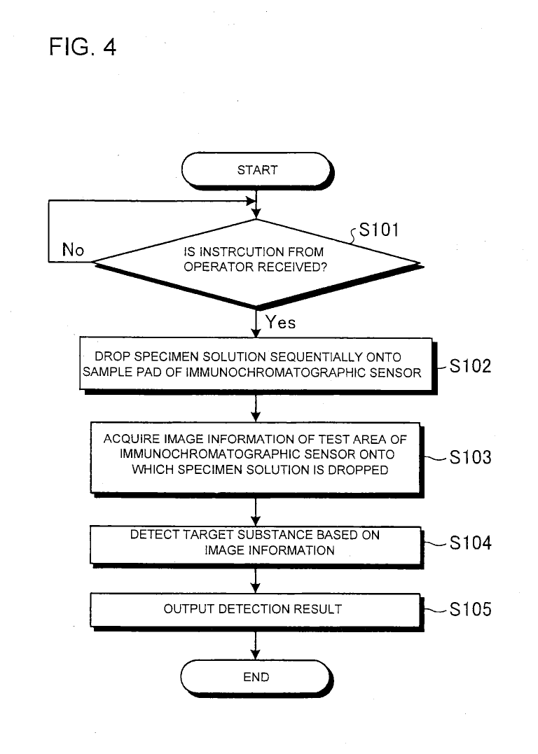

FIG. 4 is a flowchart illustrating an example of a flow of processing performed by the assay device according to the first embodiment.

As illustrated in FIG. 4, when directions from an operator are received (Yes in Step S101), in assay device 100, dropping control unit 121 drops the specimen solution sequentially onto each of sample pads 211 of immunochromatographic sensors 210 (Step S102). For example, dropping unit 102 drops the specimen solution onto the sample pad of the immunochromatographic sensor. In addition, dropping control unit 121 moves sheet 200 each time the specimen solution has been dropped so that sample pad 211 of the subsequent immunochromatographic sensor 210 will be in a position where the specimen solution is to be dropped by dropping unit 102.

Acquisition unit 122 acquires the image information of test area 213 of the immunochromatographic sensor 210 onto which the specimen solution is dropped by dropping unit 102 (Step S103). Specifically, acquisition unit 122 acquires image information of each of the immunochromatographic sensors 210 onto which the specimen solution is dropped sequentially by dropping control unit 121.

Detection unit 123 detects the presence of the target substance based on the image information acquired by acquisition unit 122 (Step S104). For example, detection unit 123 determines whether the pixel value of test area 213 is equal to or greater than the threshold. When the pixel value is determined to be equal to or greater than the threshold, it means there is target substance. Conversely, when the pixel value is determined to be smaller than the threshold, it means there is no target substance.

Detection unit 123 outputs the detection result to a user via input/output unit 101 (Step S105). For example, detection unit 123 outputs the result which immunochromatographic sensor 210 has detected the target substance.

As described above, according to the first embodiment, assay device 100 drops the specimen solution sequentially onto each of the sample pads 211 of multiple immunochromatographic sensors 210 adjacent to each other in a transverse direction, and acquires the image information of test area 213 of each of the immunochromatographic sensors 210 onto which the specimen solution is dropped. As a result, the process of continuously detecting the target substance is simplified.

According to the first embodiment, assay device 100 detects the presence of the target substance based on the image information. As a result, continuously detecting the target substance is simplified, and the user's visual check is no longer necessary on test areas 213 of multiple immunochromatographic sensors 210 adjacent to each other in a transverse direction.

According to the first embodiment, immunochromatographic sensor 210 includes sample pad 211, conjugate pad 212, test area 213, control line 214 and absorption area 215, which are arranged in that order in a longitudinal direction, and multiple immunochromatographic sensors 210 are arranged side by side in a transverse direction. As a result, an assay on multiple immunochromatographic sensors 210 adjacent to each other in a transverse direction is performed continuously in a short period of time.

According to the first embodiment, sheet 200 has intersensor area 230 between immunochromatographic sensors 210 adjacent to each other in a transverse direction. Intersensor area 230 is an area where the movement speed of the specimen solution in a transverse direction is relatively slow as compared with the movement speed in a longitudinal direction. As a result, the specimen solution, dropped onto sample pad 211 of a certain immunochromatographic sensor 210, is prevented from influencing the specimen solution dropped onto other sample pads.

According to the first embodiment, sheet 200 has a hydrophobic portion or a break as the intersensor area 230. As a result, forming intersensor area 230 is simplified.

### Second Embodiment

So far, the assay device and assay method according to the first embodiment have been described. However, the assay device and method related to the present invention are not limited to the examples above. In the following, a second embodiment will be described. Descriptions of the portions the same as in those in assay device 100 of the first embodiment will be omitted herein.

According to an example of the present embodiment, an assay device 300 further includes a forming unit which forms multiple immunochromatographic sensors 210 arranged adjacent to each other in a transverse direction on a sheet 200.

According to an example of the present embodiment, assay device 300 is structured such that the kinds of multiple immunochromatographic sensors 210, which are arranged adjacent to each other in a transverse direction on sheet 200, are changed.

### Structure of Assay Device According to Second Embodiment

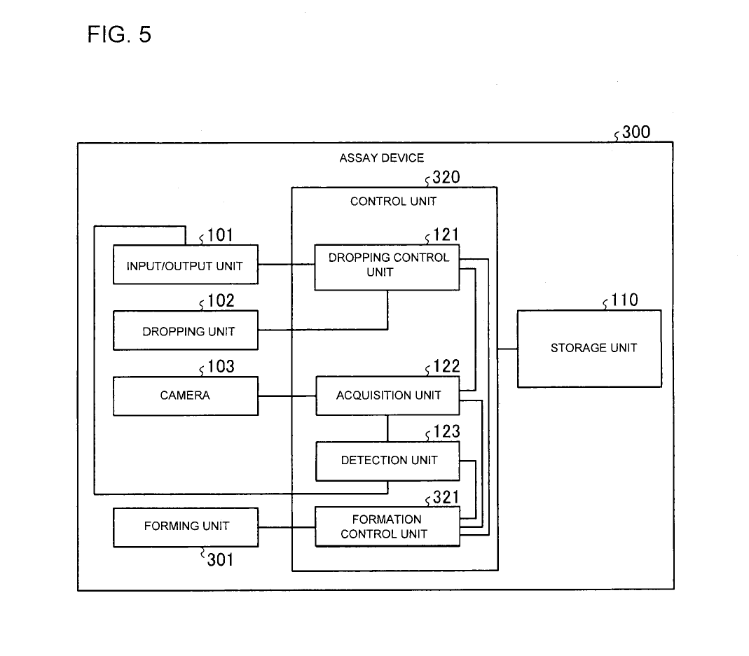

FIG. 5 is a block diagram illustrating an example of the structure of the assay device according to the second embodiment. As illustrated in FIG. 5, assay device 300 further includes a forming unit 301, and a control unit 320 further includes a formation control unit 321.

Formation unit 301 forms multiple immunochromatographic sensors 210 on sheet 200 by being controlled by formation control unit 321. Forming unit 301 is a printer, for example.

Formation control unit 321 controls forming unit 301 in order to form multiple immunochromatographic sensors 210 adjacent to each other in a transverse direction on sheet 200. For example, immunochromatographic sensors 210 are formed in such a manner that forming unit 301 prints a labeled substance and/or an antibody on sheet 200, and an intersensor area 230 is formed by printing a hydrophobic substance on or putting a break in sheet 200. In addition, in this case, before formation control unit 321 forms immunochromatographic sensors 210, immunochromatographic sensors 210 may be partially formed beforehand on sheet 200. For example, sample pads 211 and absorption areas 215 may be formed on sheet 200 beforehand. In this case, formation control unit 321 attaches the labeled substance corresponding to a target substance by printing the labeled substance on a conjugate pad 212, immobilizes an antibody which specifically binds to a complex of the labeled substance and the target substance to test area 213 by printing the antibody, and immobilizes an antibody which specifically binds to the labeled substance by printing the antibody to a control line 214.

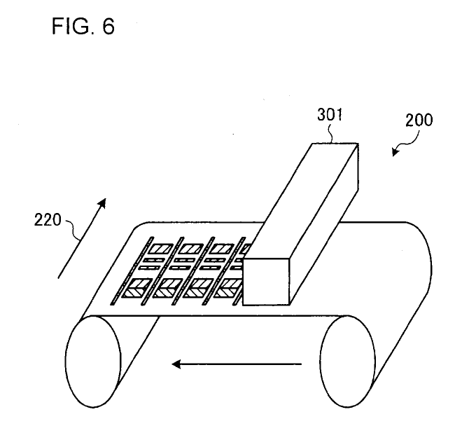

FIG. 6 is a diagram illustrating a process of forming the immunochromatographic sensors using the formation control unit according to the second embodiment. As illustrated in FIG. 6, formation control unit 321 sequentially forms immunochromatographic sensor 210, for example, by controlling forming unit 301. In the example illustrated in FIG. 6, forming unit 301 is illustrated in a rectangular form in order to simplify the description.

Formation control unit 321 may change the type of the immunochromatographic sensors 210 adjacent to each other in a transverse direction on sheet 200. In other words, formation control unit 321 may change the target substance to be detected by the immunochromatographic sensor 210. For example, formation control unit 321 may change the target substance by changing the kind of antibody immobilized on test area 213. However, that is not the only option for a method for changing the target substance, and any other technique may also be employed.

For example, it is an option for formation control unit 321 to change the target substance for detecting multiple target substances separately, or for changing the target substance in each time frame in which detection processing is performed. In addition, for example, formation control unit 321 may change the kind of immunochromatographic sensor 210 formed by forming unit 301, based on the detection results of detection unit 123. More specifically, when a target substance is detected by detection unit 123, immunochromatographic sensors 210 formed on sheet 200 may be changed for detecting another target substance.

Similarly, formation control unit 321 may change the method for forming intersensor area 230. For example, based on image information acquired by acquisition unit 122, formation control unit 321 determines whether the specimen solution spreads over onto adjacent immunochromatographic sensors 210 beyond intersensor area 230. Here, when the specimen solution is determined to have spread over onto the adjacent immunochromatographic sensors 210, formation control unit 321 changes the method for forming intersensor area 230. For example, when existing intersensor areas 230 are formed by printing a hydrophobic substance, new intersensor areas 230 will be formed by forming a break. In this case, forming unit 301 includes a cutter, an edged tool, a roller or the like for forming the break beforehand, and forms the break on sheet 200 based on the control of formation control unit 321.

As described above, according to assay device 300 of the second embodiment, because assay device 300 further includes forming unit 301 which forms multiple immunochromatographic sensors 210 adjacent to each other in a transverse direction on sheet 200, it is not necessary to prepare multiple immunochromatographic sensors 210 in advance, and immunochromatographic sensors 210 are prepared on the spot each time to perform detection processing.

According to assay device 300 of the second embodiment, the kinds of the immunochromatographic sensors 210 formed on sheet 200 can be changed. As a result, various target substances can be continuously, simply detected.

### OTHER EMBODIMENTS

So far, the first and second embodiments have been described. However, the present invention is not limited to those, and may be carried out by other embodiments. Therefore, other embodiments are described in the following.

For example, among each process described in the embodiments, all or a part of the processes which are described to be automatically performed may be manually performed, or all or a part of the processes which have been described to be manually performed may be automatically performed by a known method. In addition, the processing procedure, the control procedure, and specific names and information (refer to FIGS. 1 to 6) including various kinds of data and parameters may be arbitrarily changed unless otherwise specified.

Moreover, each structural element of each device illustrated is a conceptual and functional unit. Therefore, each structural element is not necessarily formed physically in the same way as illustrated. Namely, a concrete form of distribution or integration of each device is not limited to the illustrated form, but some or all of the units of each device may be functionally or physically integrated or distributed in arbitrary units depending on the kinds of loads or usage conditions. For example, dropping unit 102 and camera 103 may be provided as separate devices and connected to each other via a network in the example shown in FIG. 1.

For example, although the second embodiment describes a case where assay device 300 changes immunochromatographic sensors 210, assay device 300 is not limited thereto. Furthermore, formation control unit 321 may form the same kind of immunochromatographic sensors 210 on sheet 200.

For example, the embodiments above describe a case where multiple immunochromatographic sensors 210 are arranged adjacent to each other in a transverse direction on sheet 200. However, the present invention is not limited to such a case, and not all the multiple immunochromatographic sensors 210 arranged adjacent to each other in a transverse direction need to be formed on the sheet 200.

For example, in the above-described embodiments, assay device 100 detects the presence of a target substance. However, the assay device of the present invention is not limited thereto.

For example, detection processing may be performed by assay device 100 or by a different device, or may be performed manually. In such a case, it is an option for assay device 100 to output the image information acquired by acquisition unit 122 to another device that performs detection processing, or to display the image information on a monitor for visual detection.

According to one embodiment, the assay device includes a dropping unit which drops a specimen solution sequentially onto each of the sample pads of multiple immunochromatographic sensors which are arranged adjacent to each other in a transverse direction, and acquires the image information of the test area of each immunochromatographic sensor onto which the specimen solution is dropped by the dropping unit.

Using the assay device according to an embodiment, continuously detecting the target substance is simplified.

Obviously, numerous modifications and variations of the present invention are possible in light of the above teachings. It is therefore to be understood that within the scope of the appended claims, the invention may be practiced other than as specifically described herein.

What is claimed is:

1. A specimen solution assay device, comprising:
a specimen solution dropping device configured to drop a specimen solution sequentially onto each of sample pads of a plurality of immunochromatographic sensors positioned adjacent to each other in a transverse direction of each of the immunochromatographic sensors; and
an image information acquisition device configured to acquire image information of a test area of each of the immunochromatographic sensors onto which the specimen solution is dropped by the specimen solution dropping device.

2. The specimen solution assay device according to claim 1, further comprising:
a target substance detection device configured to detect the presence of a target substance based on the image information acquired by the image information acquisition device.

3. The specimen solution assay device according to claim 1, wherein each of the immunochromatographic sensors includes a sample pad, a conjugate pad, the test area, a control line and an absorption area which are positioned in an order of the sample pad, the conjugate pad, the test area, the control line and the absorption area in a longitudinal direction of each of the immunochromatographic sensors, and the plurality of immunochromatographic sensors is positioned in the transverse direction of the immunochromatographic sensors on a sheet.

4. The specimen solution assay device according to claim 3, wherein the sheet includes an intersensor area which is positioned between the immunochromatographic sensors positioned adjacent to each other in the transverse direction, and the intersensor area moves the specimen solution at a movement speed in the transverse direction which is slower than a movement speed in the longitudinal direction.

5. The specimen solution assay device according to claim 4, wherein the intersensor area of the sheet includes a hydrophobic portion or a break.

6. The specimen solution assay device according to claim 3, further comprising:
an immunochromatographic sensor-forming device configured to form the plurality of immunochromatographic sensors on the sheet.

7. The specimen solution assay device according to claim 6, wherein the immunochromatographic sensor forming device is configured to change a kind of the plurality of immunochromatographic sensors to be formed on the sheet.

8. The specimen solution assay device according to claim 2, wherein each of the immunochromatographic sensors includes a sample pad, a conjugate pad, the test area, a control line and an absorption area which are positioned in an order of the sample pad, the conjugate pad, the test area, the control line and the absorption area in a longitudinal direction of each of the immunochromatographic sensors, and the plurality of immunochromatographic sensors is positioned in the transverse direction of the immunochromatographic sensors on a sheet.

9. The specimen solution assay device according to claim 4, further comprising:
an immunochromatographic sensor-forming device configured to form the plurality of immunochromatographic sensors on the sheet.

10. The specimen solution assay device according to claim 5, further comprising:
an immunochromatographic sensor-forming device configured to form the plurality of immunochromatographic sensors on the sheet.

11. An immunochromatographic sensor device, comprising:
a sheet; and
a plurality of immunochromatographic sensors positioned adjacent to each other in a transverse direction of the sheet,
wherein each of the immunochromatographic sensors includes a sample pad onto which a specimen solution is dropped and a test area which tests the presence of a target substance in the specimen solution dropped onto the sample pad.

12. The immunochromatographic sensor device according to claim 11, wherein each of the immunochromatographic sensors includes the sample pad, a conjugate pad, the test area, a control line and an absorption area which are positioned in an order of the sample pad, the conjugate pad, the test area, the control line and the absorption area in a longitudinal direction of each of the immunochromatographic sensors, and the plurality of immunochromatographic sensors is positioned in the transverse direction of the immunochromatographic sensors on the sheet.

13. The immunochromatographic sensor device according to claim 12, wherein the sheet includes an intersensor area which is positioned between the immunochromatographic sensors positioned adjacent to each other in the transverse direction, and the intersensor area moves the specimen solution at a movement speed in the transverse direction which is slower than a movement speed in the longitudinal direction.

14. The immunochromatographic sensor device according to claim 13, wherein the intersensor area of the sheet includes a hydrophobic portion or a break.

15. A specimen solution assay method, comprising:
dropping a specimen solution sequentially onto each of sample pads of a plurality of immunochromatographic sensors which are positioned adjacent to each other in a transverse direction of each of the immunochromatographic sensors; and
acquiring image information of a test area of each of the immunochromatographic sensors onto which the specimen solution is dropped.

16. The specimen solution assay method according to claim 15, further comprising:
detecting the presence of a target substance based on the acquired image information.

17. The specimen solution assay method according to claim 15, wherein each of the immunochromatographic sensors includes a sample pad, a conjugate pad, the test area, a control line and an absorption area which are positioned in an order of the sample pad, the conjugate pad, the test area, the control line and the absorption area in a longitudinal direction of each of the immunochromatographic sensors, and the plurality of immunochromatographic sensors is positioned in the transverse direction of the immunochromatographic sensors on a sheet.

18. The specimen solution assay method according to claim 17, wherein the sheet includes an intersensor area which is positioned between the immunochromatographic sensors positioned adjacent to each other in the transverse direction, and the intersensor area moves the specimen solution at a movement speed in the transverse direction which is slower than a movement speed in the longitudinal direction.

19. The specimen solution assay method according to claim 18, wherein the intersensor area of the sheet includes a hydrophobic portion or a break.

20. The specimen solution assay method according to claim 17, further comprising:
forming the plurality of immunochromatographic sensors on the sheet.
